(19) United States
(12) Patent Application Publication
(10) Pub. No.: US 2026/0237059 A1
(43) Pub. Date: Aug. 13, 2026

(54) METHOD FOR CHARACTERIZING CARDIAC LESIONS AND ASSOCIATED SYSTEMS

(71) Applicants: FONDATION BORDEAUX UNIVERSITÉ, BORDEAUX (FR); UNIVERSITE DE BORDEAUX, BORDEAUX (FR); CENTRE HOSPITALIER UNIVERSITAIRE DE BORDEAUX, TALENCE CEDEX (FR)

(72) Inventors: Aurélien BUSTIN, LÉOGEATS (FR); Hubert COCHET, BORDEAUX (FR)

(21) Appl. No.: 19/150,796

(22) PCT Filed: Jan. 25, 2024
(86) PCT No.: PCT/EP2024/051797

§ 371 Date: Jul. 24, 2025

(30) Foreign Application Priority Data
Jan. 25, 2023 (FR) FR2300699

(51) Int. Cl. G06T 7/00; A61B 5/00; A61B 5/055; G06T 7/11; G06T 7/136; G06T 7/60; G06T 7/70

(52) U.S. Cl. CPC G06T 7/0012; A61B 5/0044; A61B 5/055; G06T 7/11; G06T 7/136; G06T 7/60; G06T 7/70; G06T 2207/10088; G06T 2207/20081; G06T 2207/20084; G06T 2207/30048; G06T 2207/30096

(57) ABSTRACT

A method for heart lesion characterization using images including a black blood image generated from signals acquired during a black blood-late gadolinium enhancement magnetic resonance acquisition, a bright blood image generated from signals acquired during a bright blood-late gadolinium enhancement magnetic resonance acquisition, the bright blood acquisition procedure being distinct from an inversion-recovery sequence, the method including computer segmenting the bright blood image so as to generate positioning data of a set of at least one wall delimiting the myocardium, and computer heart lesionally characterizing, from the black blood image and positioning data, at least one wall of the set of at least one wall, taken from the positioning data of a set of at least one wall delimiting the myocardium.

### FIELD OF THE INVENTION

The invention relates to the field of cardiac Magnetic Resonance imaging (MRI) by Late Gadolinium Enhancement (LGE).

The field of application of the invention more particularly concerns methods and systems for heart lesion characterization. This characterization especially allows guidance of ablations.

The reference technique for the characterization of regional lesions including myocardial fibrosis is imaging by BRight blood-Late Gadolinium Enhancement (BR-LGE) by inversion-recovery such as the PSIR (Phase-Sensitive Inversion-Recovery”) sequence. In this type of imaging, cancelation of the viable myocardial signal is brought about using inversion-recovery pulses, which allows lesions to be visualized with high contrast between healthy myocardial tissue and lesions. However, for myocardial lesions adjacent to the blood cavities of the heart (right and left ventricles), the high intensity of the signal from blood and therefore low contrast between lesions and blood prevents automatic, accurate, reliable and robust characterization of scars, in particular subendocardial scars.

In order to circumvent this problem, BLack blood LGE (BL-LGE) imaging techniques have been provided. They make it possible to cancel out signals from healthy myocardium and blood simultaneously, thereby providing high contrast between both lesions and blood and between lesions and healthy myocardium.

However, black blood imaging techniques do not allow properly characterizing lesions, including locating them accurately relative to the myocardium, as the contrast between blood and healthy myocardium is not high enough.

In view of the aforementioned drawbacks, scar characterization is currently carried out by radiologists by manually segmenting lesions from images derived from the PSIR sequences, which then makes it possible to calculate their transmurality and size. However, this method is time-consuming since it takes the radiologist 25 to 30 minutes to perform such segmentation. Furthermore, such segmentation is inaccurate and not reproducible. Indeed, the low contrast between lesion and blood, often leading the radiologist to imagine the subendocardial wall, which leads to an overestimation or underestimation of characteristics of the scar.

One purpose of the invention is to limit at least one of the aforementioned drawbacks.

To this end, one object of the invention is a method for characterizing heart lesion using images of a zone to be imaged comprising the heart of a patient, the heart comprising a myocardium delimiting a cavity of the heart, the images comprising:
a black blood image generated from signals acquired during an acquisition by black blood-late gadolinium enhancement magnetic resonance,
a bright blood image generated from signals acquired during an acquisition by bright blood-late gadolinium enhancement magnetic resonance, the bright blood magnetic resonance acquisition being distinct from an inversion-recovery sequence,
said method comprising:
computer segmenting the bright blood image so as to generate positioning data of a set of at least one wall delimiting the myocardium,
computer heart lesionally characterizing, from the black blood image and positioning data, at least one wall of the set of at least one wall, taken from the positioning data of a set of at least one wall delimiting the myocardium.

Advantageously, segmenting uses a learning function to segment the bright blood image so as to obtain the positioning data.

Advantageously, the learning function is a convolutional neural network.

Advantageously, the learning function is trained from a set of training images of the zone to be imaged generated from signals acquired during respective acquisition steps by bright blood-late gadolinium enhancement magnetic resonance distinct from inversion-recovery sequences.

Advantageously, the set of at least one wall comprises a first wall delimiting and surrounding the myocardium.

Advantageously, characterizing comprises lesional segmentation to locate a myocardial lesion on the black blood image using data from first wall positioning data.

Advantageously, the lesional segmentation comprises selecting the pixels of the black blood image having an intensity greater than a predetermined threshold, the pixels being taken only from the pixels of the black blood image that are surrounded by the first wall.

Advantageously, the set of at least one wall comprises a second wall delimiting the myocardium and surrounded by the first wall.

Advantageously, characterizing comprises calculating a piece of data representative of a lesion size from data derived from the positioning data of the first wall and possibly of a second wall surrounded by the first wall.

Advantageously, characterizing comprises calculating data representative of a transmurality percentage of the lesion from data derived from the positioning data of the first wall and the second wall.

Advantageously, the method comprises displaying, on a screen, a representation of data calculated during the characterization step.

Advantageously, the method comprises:
black blood-late gadolinium enhancement magnetic resonance acquisition,
generating the black blood image from the signals acquired during the black blood-late gadolinium enhancement magnetic resonance acquisition, referred to as bright blood acquisition.
bright blood-late gadolinium enhancement magnetic resonance acquisition,
generating the bright blood image from the signals acquired during the black blood-late gadolinium enhancement magnetic resonance acquisition, referred to as black blood acquisition.

Advantageously, the black blood acquisition and the bright blood acquisition belong to an acquisition sequence comprising an elementary acquisition sequence each comprising a bright blood acquisition and a black blood acquisition.

Advantageously, in each elementary acquisition sequence, black blood acquisition and bright blood acquisition are implemented during a pair of interbeats consisting of two consecutive interbeats.

Advantageously, the elementary acquisition sequences are implemented during pairs of consecutive respective interbeats.

Advantageously, the method comprises lesional segmenting and characterizing from black blood and bright blood images generated from bright blood images and black blood images generated from signals acquired during elementary sequences of the acquisition sequence.

Advantageously, the method comprises the acquisition sequence.

Advantageously, the method comprises generating the black blood image and the bright blood image.

Advantageously, the black blood image and the bright blood image are two-dimensional.

Another object of the invention is a system comprising the hardware and software elements to implement the method according to the invention.

The system advantageously comprises a processing unit configured to implement the segmentation step and the lesion characterization step.

Advantageously, the processing unit is a processing unit.

Advantageously, the system comprises a set of measurement equipment comprising a magnetic resonance imaging device able to implement the black blood magnetic resonance acquisition and bright blood magnetic resonance acquisition.

Advantageously, the processing unit is configured to generate commands intended for the MRI device so that it implements black blood magnetic resonance acquisition and bright blood magnetic resonance acquisition.

Alternatively and/or additionally, the processing unit is configured to generate the bright blood and black blood images from the signals acquired during the respective acquisitions.

Advantageously, the system comprises an electrocardiograph configured to acquire an electrocardiogram of the patient during the respective acquisitions.

The invention also concerns a computer program product comprising instructions which cause the system according to the invention to execute the steps of the method according to the invention.

The invention also concerns a computer-readable medium, having the computer program according to the invention recorded thereon.

### BRIEF DESCRIPTION OF THE FIGURES

Other characteristics and advantages of the invention will become clearer upon reading the following detailed description, in reference to the appended figures, that illustrate:

FIG. 1: an example embodiment of a system according to the invention,

FIG. 2: a schematic representation of an elementary sequence of acquiring signals to generate black blood images and bright blood images used in the method according to the invention,

FIG. 3: a schematic representation of a black blood and bright blood MRI acquisition phase performed on a plurality of heart beats,

FIG. 4: a schematic representation of a three-dimensional (3D) heart illustrating different sectional planes distributed along the major axis of the heart and images generated from signals acquired in one of the sectional planes,

FIG. 5: a flowchart of an example method according to the invention,

FIG. 6: a schematic representation of four images comprising at the top left a bright blood image and at the top right a bright blood image on which the walls detected during the segmentation step are represented, and at the bottom left a black blood image and the representation of the walls transferred to the black blood image,

FIG. 7: at the top, the lower-left image of FIG. 6 on which sectors have been represented, at the bottom-left a Bull's eye type representation of the lesional size and at the bottom-right a Bull's eye type representation of a lesional transmurality percentage.

### DESCRIPTION OF THE INVENTION

The invention concerns the field of cardiac magnetic resonance imaging or MRI, black blood-and bright blood-late gadolinium enhancement.

The invention relates to a method for cardiac lesion characterization, and more specifically for least one cardiac muscle, for example the myocardium.

Cardiac lesion is intended to mean a lesion of a muscle of the myocardium.

Cardiac lesions can be divided into acute lesions resulting from acute myocardial condition, such as acute phase myocardial infarction, and chronic lesions characteristic of chronic cardiac pathologies. These lesions are cardiac lesions, for example of the myocardium or papillary muscles. These lesions comprise myocardial fibroses that frequently develop in the context of hypertrophic or dilated cardiomyopathies, but are also frequent sequelae of inflammatory cardiopathy or myocardial infarction.

Lesions also comprise myocardial necroses, namely the volumes of myocytes whose cell membrane has been destroyed and the volumes of the extracellular and collagen matrices making up the fibrous scars, in the chronic phase of infarction.

### Imaging System

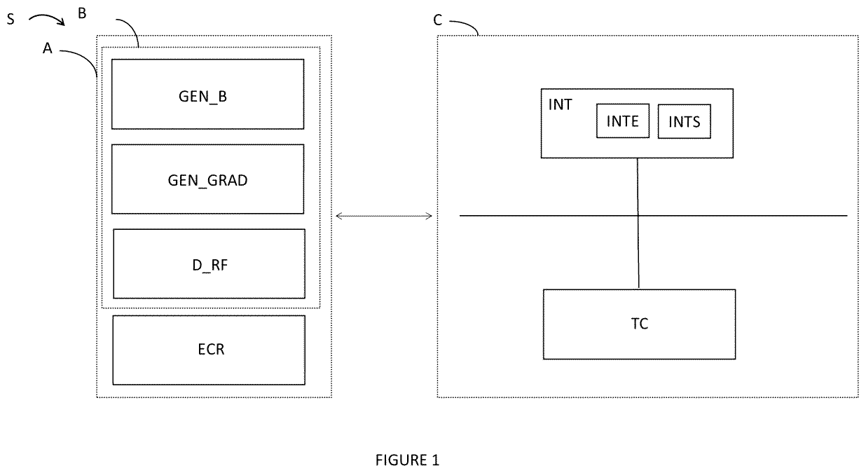

FIG. 1 schematically represents an example embodiment of a system S according to the invention. The system comprises the hardware and software means to implement the method according to the invention.

Advantageously, this system S comprises a set of measurement equipment A comprising a magnetic resonance imaging (MRI) device B as well as an electrocardiograph referenced ECR in FIG. 1.

The system S also comprises a processing device C comprising a processing unit TC and a human-machine interface INT. This processing device may be part of the imaging device B or be external to this device, the system then being a device. Alternatively, the system has a distributed architecture.

In a known manner, the MRI imaging device B comprises a static magnetic field generator GEN_B, a gradient generator GEN_GRAD and a radiofrequency (RF) device D_RF.

The static magnetic field generator GEN_B comprises a main polarization magnet for generating a substantially uniform polarization static magnetic field in a polarization zone (generally a tunnel) for comprising the zone to be imaged of the patient, this zone to be imaged comprising the heart.

The patient is a mammal. In a non-limiting manner, the mammal is a man.

The gradient generator GEN_GRAD comprises three gradient coils (or solenoids) disposed and configured to vary intensity of the magnetic field in the polarization zone along respective orthogonal axes x, y, and z fixed with respect to the polarization zone. The choice of intensities circulating in these coils makes it possible to select, from several possibilities, a cross-section, having a thickness and a sectional plane on which the cross-section is centered, in which the magnetization of the zone to be imaged of the patient received in the polarization zone will be measured.

The radiofrequency device GEN_RF comprises coils or solenoids and is able to generate MRI acquisition sequences comprising magnetization preparation sequences of the zone to be imaged and sequences of reading RF signals derived from the zone to be imaged.

Each preparation and read sequence comprises at least one radiofrequency pulse of predetermined and adjustable frequency, shape, duration, phase, amplitude.

The preparation sequence is configured to excite, namely change direction of magnetization of the tissues of the zone to be imaged.

The read sequence is configured to measure magnetization of the zone to be imaged resulting from the preparation module.

The electrocardiograph ECR is to acquire an electrocardiogram of the patient.

The processing unit TC is configured to generate commands intended for the MRI device B, especially intended for the RF device D_RF and the gradient generator GEN_GRAD, so that the MRI device generates the predefined acquisition sequences of signals derived from predefined volumes or cross-sections of the zone to be imaged.

The processing unit CT is also configured to generate images of the zone to be imaged from the measured signals, from reconstruction techniques known to those skilled in the art, and to process these images as will be seen in more detail in the remainder of the description.

### Acquisition Sequence

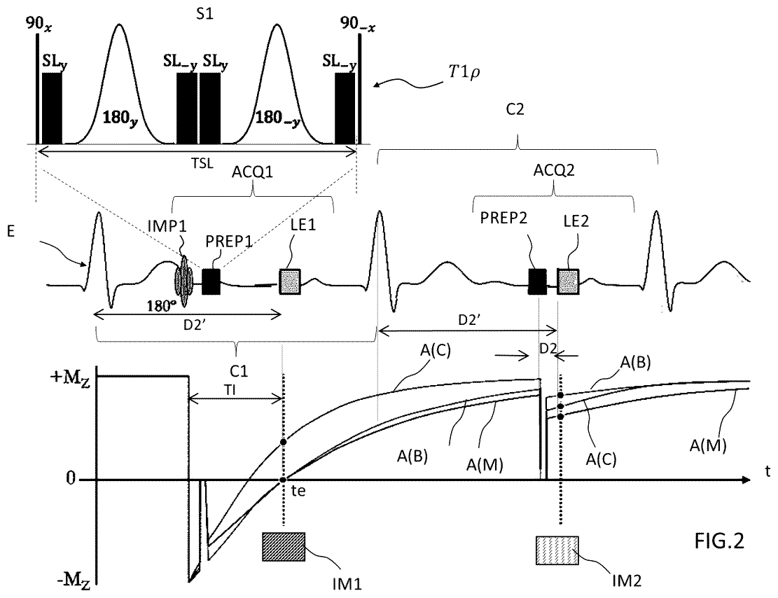

FIG. 2 shows an example of elementary acquisition sequence SE1 of an MRI acquisition sequence of RF signals for generating images of the heart as well as an electrocardiogram (ECG) E measured by the electrocardiograph ECR during the elementary sequence SE1.

The acquisition sequence comprises a series of elementary acquisition sequences SE1 such as that represented in FIG. 2.

The lower part of FIG. 2 represents the variation in the longitudinal magnetization Mz of the tissues of the zone to be imaged as a function of time t during this elementary sequence SE1.

The elementary acquisition sequence SE1 comprises a so-called black blood acquisition ACQ1 followed by a so-called bright blood acquisition ACQ2 which will be described subsequently. Black blood acquisition ACQ1 makes it possible to acquire the signals of the zone to be imaged for generating an elementary black blood image IM1 of the zone to be imaged. Bright blood acquisition ACQ2 makes it possible to acquire the signals of the zone to be imaged for generating an elementary bright blood image IM2 of the zone to be imaged. These acquisition steps ACQ1, ACQ2 each comprise a preparation module PREP1, PREP2 and a read module LE1, LE2.

In the present patent application, by module, it is meant a step comprising a radiofrequency pulse or a series of radiofrequency pulses.

It should be noted that during the entire duration of the elementary acquisition sequence SE1 and preferably during the entire duration of the MRI acquisition sequence, the static magnetic field generator GEN_B is controlled by the processing unit TC so that it generates a fixed static magnetic field along the axis z.

For its part, the gradient generator GEN_GRAD is controlled for the processing unit TC so that the radiofrequency device D_RF acquires signals from a predefined cross-section having a predefined thickness during the elementary acquisition sequence SE1.

The acquisition sequence is an acquisition sequence by late gadolinium enhancement implemented following the injection of a gadolinium-based contrast agent intravenously into the patient, from 10 to 15 minutes before the implementation of the acquisition sequences so as to obtain images having maximum contrast between the lesions and healthy tissue and blood. At the heart, the contrast is rapidly removed from healthy myocardium, which is low in interstitial tissue, but accumulates in a prolonged manner in myocardial lesions. Gadolinium has an extracellular distribution, namely it does not cross cardiomyocyte membranes.

Gadolinium has the effect of shortening the relaxation time T1 of the tissue where it accumulates. The relaxation of magnetization of lesions following a magnetization inversion pulse is thus faster than that of blood and healthy myocardium.

### Black Blood Acquisition

Firstly, it is sought to generate elementary black blood images IM1. In an image of this type, the intensity of the pixels corresponding to blood and healthy muscle is zero (black pixels) or substantially zero.

In order to generate such an elementary black blood image IM1, the RF device D_RF implements a black blood acquisition step ACQ1 in inversion-recovery. This acquisition black blood sequence ACQ1 comprises a longitudinal inversion pulse noted 180° in FIG. 2, which turns the longitudinal magnetization of the tissues of the zone to be imaged to the opposite direction, namely which inverses longitudinal magnetization of this tissue. In FIG. 2, it is noticed that magnetization of the zone to be imaged changes from Mz to −Mz under the effect of the inversion pulse. Due to the longitudinal relaxation, the longitudinal magnetization of the different tissues present in the zone to be imaged increases to return to its initial value, passing through the null value. Naturally, the relaxation kinetics of different tissues are different.

In a manner known per se, the black blood acquisition ACQ1 also comprises a preparation module PREP1 implemented after the 180° longitudinal inversion pulse, for example, a T1-rho adiabatic module (T1p) having duration noted TSL (“Time of Spin Lock”) or a T2-weighted or MTC (“Magnetization Transfer Contrast”)-type module or a combination of two of these modules or of these three modules.

The preparation module PREP1 is configured so that longitudinal magnetization of blood B (Blood) and that of healthy myocardium A (Musc) are canceled out at the same instant te.

At this same instant te, longitudinal magnetization of the lesions A (cica) is significantly greater than zero. By acquiring signals derived from the zone to be imaged at this instant te, an image is obtained with a very high contrast between the pixels or voxels corresponding to blood and healthy myocardium, which are black, and the pixels or voxels corresponding to lesions, which are overall bright.

The first acquisition step ACQ1 in inversion-recovery then comprises a read sequence LE1 comprising a 90° pulse applied at the instant te and a read gradient to read transverse magnetization of the zone to be imaged. The inversion time TI is the duration separating the 180° pulse from the read sequence LE1 of the elementary sequence ACQ1. In order to obtain the best contrast between myocardial lesions and blood as well as between myocardial lesions and healthy myocardium, the read sequence LE1 is advantageously started at instant te where longitudinal magnetizations of blood and myocardium cancel out in order to generate the image exhibiting the best contrast.

In the example of FIG. 2, the read module LE1 of the black blood acquisition is temporally spaced from the preparation module PREP1 of the black blood acquisition. Alternatively, the read module LE1 starts as soon as the preparation module PREP1 ends. The same applies to the relative time positioning between the preparation module PREP2 of the bright blood acquisition and the read module LE2 of the bright blood acquisition.

In the example of FIG. 2, the inversion pulse IMP1 is generated before the preparation module PREP1. Alternatively, the preparation module PREP1 is generated before the inversion pulse IMP1.

### Bright Blood Acquisition

The elementary bright blood image IM2 of the zone to be imaged is generated from signals acquired by implementing the bright blood acquisition step ACQ2 comprising a preparation module PREP2 followed by a read module LE2.

Advantageously, the preparation module PREP2 is identical to the preparation module PREP1 of the black blood acquisition step ACQ1, but the invention also applies when these modules are distinct.

The preparation module PREP2 is, for example, a T1rho adiabatic sequence.

Alternatively, the module PREP2 comprises at least one preparation sequence taken from a T2-weighted module and a preparation module of the MTC (“Magnetization Transfer Contrast”) type or a combination of two of these modules or of these three modules.

The bright blood acquisition step ACQ2 then comprises a read module LE2 comprising a reading gradient to read transverse magnetization of the zone to be imaged. This read module LE2 may be made in gradient echo or spin echo, just like the read module LE1 of the black blood acquisition step LE1. The read modules LE1 and LE2 can be identical or different.

The duration D2 separating the read module LE2 is defined such that the longitudinal magnetization of blood A(Blood) is greater than that of Myocardium A(MUSC) which leads to generating an image in which the pixels or voxels of the blood are bright, namely with high luminance, and in which the pixels of the tissues of the myocardium are slightly less luminous than those of the blood as can be deduced from the curves represented in FIG. 2. These images allow for a perfect visualization of the heart anatomy for delimiting the myocardium on this image, which is not possible on a black blood image.

It is understood that by using the two images IM1 and IM2, it is possible to detect this lesion and also to characterize it, for example to locate it accurately relative to the myocardium and to dimension it relative to the myocardium.

### Synchronization of Acquisition Steps With Cardiac Cycles

Preferably, the processing unit TC is configured to synchronize the acquisition sequence SE with the electrocardiogram E.

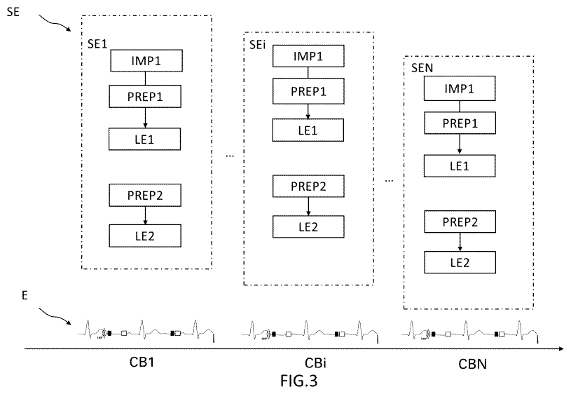

To this end, the processing unit TC uses the electrocardiogram E to generate commands for triggering acquisition sequences intended for the RF device, the gradient generator and possibly the main magnetic field generator. Advantageously, the acquisition sequence SE comprises, as visible in FIG. 3, a plurality of elementary acquisition sequences SEi, with i=1 to N, where N is greater than 1 where i is the index of the elementary sequence, the acquisition steps ACQ1, ACQ2 of which are identical. i=1 in FIG. 2.

Each elementary acquisition sequence SEi is advantageously implemented during two consecutive cardiac cycles, preferably during two consecutive interbeats C1, C2 referenced in FIG. 2 constituting a pair of interbeats CBi referenced in FIG. 1. One interest is to minimize acquisition time and therefore to minimize movements of the heart between the different acquisitions and spatial shifts between the images IM1 and IM2.

In the remainder of the text, a beat refers to a QRS complex, and an interbeat refers to a phase of a cardiac cycle between two consecutive beats.

Advantageously, the consecutive elementary acquisition sequences SEi are implemented during pairs of consecutive interbeats CBi.

Each elementary acquisition sequence SEi comprises:
During the first interbeat C1 of the pair of interbeats CBi, the black blood acquisition step ACQ1;
During the second interbeat C2 of the pair of interbeats CBi, the bright blood acquisition step ACQ2.

One interest is to minimize acquisition time and therefore to minimize movements of the heart between the different acquisitions and the spatial shifts between the images IM1 and IM2 acquired during the different elementary sequences SEi.

Advantageously, as represented in FIG. 2, the acquisition sequence SE and the electrocardiogram E are synchronized such that the read modules LE1, LE2 are implemented during a same phase of respective cardiac cycles C1, C2.

Advantageously, this phase is an interbeat.

Advantageously, this phase is diastole.

Advantageously, the acquisition sequence SE and the electrocardiogram E are synchronized such that the read modules LE1, LE2 of the black blood and bright blood acquisition steps ACQ1, ACQ2 are implemented at same instants of these respective cardiac cycles C1, C2.

These instants are defined in relation to a same time reference of the cardiac cycles C1, C2. The time reference is, for example; the maximum of the QRS complex.

These instants are separated by a same duration D2′ from the maximum of the R wave in the example of FIG. 2.

Synchronization of the read modules LE1, LE2 of the black blood and bright blood acquisition sequences ACQ1, ACQ2 and, as will be seen later, of different read modules of bright blood acquisition steps on the one hand and the different read modules of black blood acquisition steps on the other hand, allows generating images of the heart at instants when the heart occupies the same position in a fixed reference frame with respect to the main magnet, which makes it possible to superimpose the images obtained without the need for a registration or by performing a simple registration.

The invention also applies when the order of the acquisition steps is different. For example, it is possible to implement several black blood acquisition steps ACQ1 during consecutive interbeats and then several bright blood acquisition steps ACQ2 during consecutive interbeats and vice versa.

It is also possible to implement at least one black blood acquisition step ACQ1 and at least one bright blood acquisition step ACQ2 during a same interbeat.

Alternatively, at least one bright blood acquisition step ACQ2 is spaced from a black blood acquisition step ACQ1 nearest in time.

### Acquisition of Cross-sections

Advantageously, the processing and control unit TC is configured so as to generate commands intended for the MRI device to acquire signals of respective cross-sections distributed along a predefined axis of the heart.

The axis is advantageously the major axis of the heart. The cross-sections obtained are then so-called minor axis cross-sections. One advantage is to allow excellent visualization of both ventricles. However, the invention also applies to the case where the cross-sections are distributed along another axis of the heart, for example a 2-cavity axis (vertical long axis) or 4-cavity axis (horizontal long axis) of the heart. Each cross-section has a defined thickness along the axis and is centered on a predefined sectional plane perpendicular to the axis. In the present application, by cross-section, it is meant a slice or layer perpendicular to the axis g and having a predefined thickness along the axis.

Advantageously, the cross-sections are contiguous along the axis.

This is achieved by the commands generated by choosing the commands generated for the gradient generator GEN_GRAD by synchronizing the commands intended for the gradient generator GEN_GRAD and intended for the RF device D_RF.

Advantageously, the signals are acquired along adjoining or partially overlapping cross-sections. This allows the entire heart to be imaged.

Advantageously, the gradient generator GEN_GRAD is controlled so that it is possible to generate several two-dimensional images of each cross-section, from the signals acquired during the acquisition sequence SE.

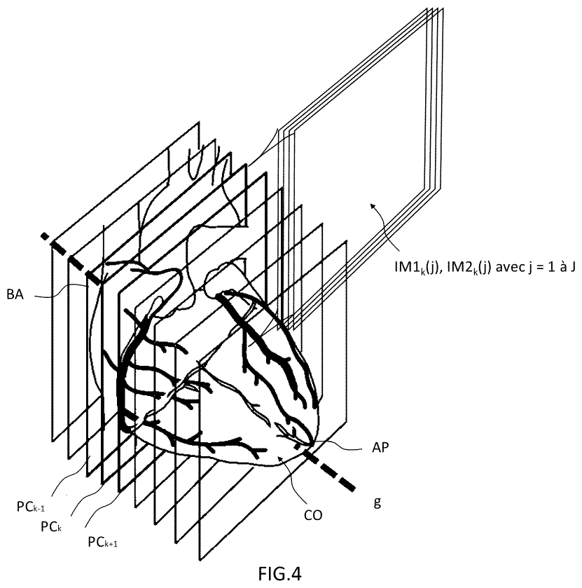

FIG. 4 shows a three-dimensional view (3D) of a heart including a plurality of sectional planes noted PCₖ distributed along the major axis g, with k=1 to K, K being an integer greater than 1. The sectional planes are distributed from the apex AP to the base BA of the heart CO. Consequently, the cross-sections are so-called “minor axis cross-sections”.

Only the images derived from the cross-section centered on the sectional plane PCₖ are represented in FIG. 4.

Preferably, several black blood elementary images IM1ₖ (j) and/or several bright blood elementary images IM2ₖ (j) are generated with j=1 to J, J being an integer greater than or equal to 2 for at least one sectional plane PCₖ, for example for each sectional plane PCₖ. This makes analysis of the images obtained more robust.

Alternatively, the system is configured to acquire signals coming from a volume, for example from the entire heart so as to enable generation of three-dimensional images.

Advantageously, the acquisition sequence SE is implemented while the patient is in apnea. One advantage is to obtain perfectly registered images which make it possible to limit, simplify or dispense with image registration.

Alternatively, the acquisition sequence SE is implemented in free-breathing. Free-breathing acquisition has time advantages. Indeed, apnea acquisition has to be quick, which results in the need to reduce the acquisition time from several seconds to several minutes and often involves having to reduce the zone to be imaged, for example by limiting it to a 3D portion. Well, since the acquisition sequence SE is synchronized with the ECG so that the read sequences are implemented at a same time marker of different cardiac cycles, the apnea acquisition leads to a limited number of images being generated.

### Image Generation

The processing unit TC is configured to generate GEN, by reconstruction techniques known to those skilled in the art, images of the zone to be imaged.

For example, the GRAPPA algorithm or the SENSE algorithm (and its iterative version) can be used.

The generation step comprises a step of generating the two-dimensional (2D) or three-dimensional (3D) elementary images of the zone to be imaged from the signals acquired during the acquisition sequence SE.

In the 2D case, advantageously, for each black blood acquisition step ACQ1, a black blood image IM1 is generated from the signals acquired during the black blood acquisition step ACQ1 and, for each bright blood acquisition step ACQ2, a bright blood image IM2 is generated from the signals acquired during the bright blood acquisition step ACQ2.

The elementary images IM1, IM2 are advantageously generated in gray levels. Each image comprises a set of pixel or voxel type unit elements each characterized by an intensity I likely to take a set of N values (N being a finite integer greater than 1) corresponding to N gray levels ranging from 0 to N−1. For example, this value may take 256 values between 0 and 255 but N is not limited to 256. This value may advantageously take 4096 values between 0 and 4095.

The processing unit CT is also configured to use the elementary images obtained to characterize the lesions of the heart, for example the myocardium as will be seen in more detail in the remainder of the text.

The elementary images generated by the processing unit TC may be intended to be displayed on a screen of the human-machine interface INT.

### Registration

Advantageously, the method is free of an image registration step.

Alternatively, the image generation step GEN comprises registering elementary black blood images between them and/or registering elementary bright blood images between them and/or registering elementary black blood and bright blood images between them. This makes it possible, especially when the patient is in free-breathing during the acquisition sequence SE, to limit effects of breathing on the position of the heart and therefore to avoid spatial shifts induced by breathing on the images and likely to affect accuracy and reliability of analyses of these images or combination of these images. Indeed, the rhythm of breathing is a priori different from the heart rhythm, but even if correlations exist between these two rhythms, it is possible that breathing accelerates while the heartbeat remains stable or vice versa.

Advantageously, the method comprises registering elementary black blood images of a same cross-section.

Advantageously, registering is carried out implementing a non-rigid image registration algorithm.

Advantageously, the method comprises registering elementary bright blood images of a same cross-section.

Advantageously, registering is carried out implementing a non-rigid image registration algorithm.

Advantageously, the method comprises registering elementary black blood IM1 and bright blood IM2 images between them.

Advantageously, the method comprises registering elementary bright blood images and elementary black blood images of a same cross-section.

Advantageously, this registering is carried out implementing a non-rigid image registration algorithm.

Advantageously, these algorithms are identical. It is possible to choose a different algorithm for processing elementary black blood and bright blood images, but it is preferable to choose the same algorithm for ease of implementation. Registration substantially improves quality, in particular contrast, of an image derived from a plurality of elementary images of a same cross-section. In addition, it reduces breathing-related artifacts.

According to a first example, at least one of the non-rigid algorithms is an algorithm based on the inter-image mutual information method based on statistical relationships. The function to be optimized may be implemented by a statistical similarity criterion. One benefit of this method is that the pairing between homologous attributes of images of a same cross-section is independent of their geometric position. Furthermore, this method is particularly effective for registering elementary images with different contrasts, such as black blood and bright blood images.

According to a second example compatible with the first example, at least one of the non-rigid algorithms based on a transformation model is implemented. The transformation model makes it possible to determine functions enabling the deviation between two images to be minimized. The deviation may be translated into a geometric error to be minimized. Different approaches may be used such as those based on extracting from each of the images geometric primitives or shape descriptors such as protruding points, shape singularities or contours. A parametric or non-parametric approach may be used.

According to one example of optimization of a transformation model or a similarity criterion, the least squares method may be used.

Other optimization methods may be implemented such as gradient descent. However, the latter method applies more particularly to image intensities and is not optimal in the scope of the invention since it is sought to optimize sharpness and contrast of the merged image. Nonetheless, the invention includes this embodiment.

The registration may be performed by choosing a reference image and determining a transformation function for the other images of the same cross-section in relation to that image. Each image is then registered by optimizing a transformation to obtain the reference image according to a geometric criterion from the image considered.

When acquiring three-dimensional images, it is possible to acquire several three-dimensional images of the heart, which will be possibly registered.

### Image Combination

When a plurality of elementary images of a same cross-section are generated, it is possible to perform operations aimed at combining, namely merging these images in order to produce a single combined image per cross-section.

Advantageously, the image generation step GEN comprises, for at least one cross-section with index k, for example for each cross-section, the combination of bright blood images of the cross-section IM₂k (j), for example for j=1 to J so as to obtain a combined bright blood image ISBₖ of the cross-section.

Advantageously, the method comprises, for at least one cross-section, for example for each cross-section, the combination of black blood images IM₁k (j), for example for j=1 to J of the cross-section with index k so as to obtain a combined black blood image ISNₖ of the cross-section.

This reduces noise and increases the signal-to-noise ratio.

The image combination can be performed before, during or after generating GEN the images.

According to one example, the combination is averaging. Averaging is, for example, performed in the image space or in the Fourier space (namely, the frequency domain, before reconstructing the images). These solutions are fast and not computationally intensive.

Averaging has the advantage of keeping detail of the image, since it increases the signal to noise ratio (SNR). This technique makes it possible to smooth out noise to reduce residual image artifacts. Further, averaging makes it possible to improve the bit depth of the digital image beyond what is possible with a single image.

One benefit of the step of averaging images made of a same cross-section is to reduce the maximum deviation. The amplitude of the noise decreases as the square root of the number of images used, namely with only 4 images, the amplitude of the noise can be reduced by a factor of two. According to an example of an acquisition in free-breathing of a duration of 2 min, it is possible to collect 4 to 5 images per sectional plane, which makes it possible to obtain good noise reduction performance.

In one example embodiment, the image combination may alternatively be implemented by motion-compensated iterative reconstruction. Stated differently, this type of combination is implemented during image reconstruction. Compensated MRI reconstruction techniques are especially described in the following articles: Odille F, et al., “Generalized reconstruction by inversion of coupled systems (GRICS) applied to free-breathing MRI” Magnetic Resonance in Medicine, 2008; et “3D whole-heart isotropic sub-millimeter resolution coronary magnetic resonance angiography with non-rigid motion-compensated PROST”, Bustin A, et al, Journal of Cardiovascular Magnetic Resonance, 2020.

Advantageously, the elementary images on the one hand of black blood and on the other hand of bright blood are respectively combined so as to produce a combined black blood image ISNₖ and a combined bright blood image ISBk per cross-section.

When acquiring three-dimensional images, it is possible to acquire several three-dimensional images of the heart, which can be combined, for example averaged, to obtain a single three-dimensional image of the heart.

### Lesion Characterization

As seen previously, during the acquisition sequence SE, signals are acquired for generating black blood elementary images IM1 and bright blood elementary images IM2 and these elementary images IM1, IM2 are generated from the signals acquired.

According to the invention, these elementary images IM1, IM2 are generated from signals acquired by implementing an acquisition sequence. This acquisition sequence comprises bright blood acquisition steps ACQ2. Each bright blood acquisition step ACQ2 is distinct from an inversion-recovery sequence.

In other words, this sequence is free of an inversion pulse of longitudinal magnetization of the zone to be imaged.

Therefore, unlike PSIR imaging during the bright blood acquisition, longitudinal magnetization of the myocardium is not canceled, thereby obtaining images with a higher contrast between lesions and blood and thus promoting diagnosis and image processing.

Advantageously, the bright blood acquisition is configured so that when implementing the read module LE2, respective longitudinal magnetizations of the healthy myocardium, blood and lesions are positive and that longitudinal magnetization of the lesions is located between magnetization of the healthy myocardium and that of blood. This is achieved by the configuration of the preparation module PREP2 and that of the read module LE2 and by the relative time positioning between these two modules.

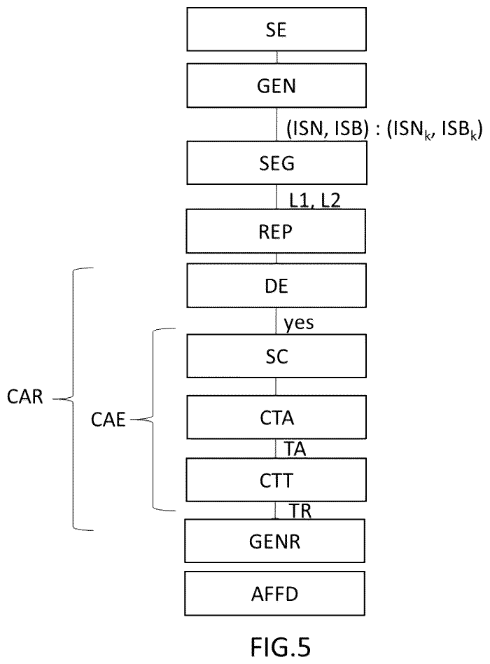

As visible in FIG. 5, the lesion characterization method comprises the following steps:
computer segmenting SEG at least one bright blood image ISB so as to generate positioning data of a first wall L1 delimiting the myocardium,
computer lesion characterizing CAR a cardiac lesion by using at least one black blood image ISN and positioning data of the first wall L1 taken from the data calculated during the segmentation step SEG.

These steps are implemented by the processing unit TC which uses for this purpose bright blood ISBₖ and black blood ISNₖ images generated by the method described previously.

Each bright blood ISB, respectively black blood ISN image is an elementary image IM₁k (j), IM₂k (j) or a combined bright blood ISBₖ, respectively black blood ISNₖ image.

In the remainder of the text, it is considered, as in the example of FIG. 5, that each bright blood image ISB is a combined bright blood image ISBₖ and that each black blood image ISN is a combined black blood image ISNₖ.

The invention makes it possible to automatically, reproducibly, reliably and accurately characterize a cardiac lesion, and more precisely of the cardiac muscles, in particular myocardial lesions.

Indeed, segmenting bright blood images ISBₖ generated from signals measured during bright blood acquisition steps ACQ2 distinct from inversion-recovery sequences allows for automatic, robust, reliable and accurate positioning of the walls delimiting the myocardium, as these images have a significant contrast between myocardium and blood. Black blood images ISNₖ dot not make it possible to obtain as good results due to the lack of contrast between healthy myocardium and blood.

Lesion characterization using, in addition to the positioning data derived from segmentation, a black blood image ISNₖ, allows obtaining good results that would not be obtained by the bright blood image ISBₖ alone containing little or no information on cardiac lesions.

### Segmentation

According to one example, segmenting SEG is implemented using at least one bright blood image ISBₖ so as to generate positioning data of a first wall L1 and a second wall L2 delimiting the myocardium M and surrounding and delimiting a cavity of the heart, the first wall L1 surrounding the second wall L2.

The positioning data relating to a wall correspond, for example, to the identification of the pixels constituting the wall.

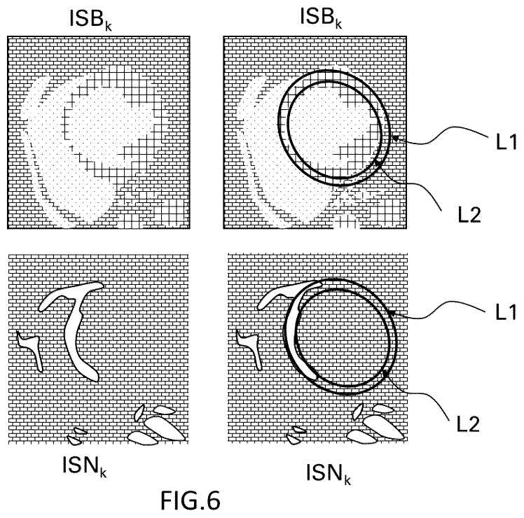

The result of this segmentation is visible in FIG. 6 schematically representing at the top left a bright blood image of a cross-section of the heart ISBₖ and at the top right the bright blood image of the cross-section on which the first wall L1 and the second wall L2 obtained during the segmentation step are represented in thick black lines.

The generated images are, for example, in gray levels. The white zones in FIG. 6 represent lighter zones than the dotted zones that represent lighter zones than the cross-hatched zones that represent lighter zones than the bricks.

In the example of FIG. 6, the heart cavity is the left ventricle and segmenting SEG is implemented so as to delimit the walls L1, L2 of the part of the myocardium surrounding and delimiting the left ventricle.

It should be noted that in the present description the invention is described in the case where the cavity of the heart is the left ventricle, but the invention is applicable to any cavity of the heart, such as the right ventricle and the atria which are also surrounded and delimited by the myocardium and subject to cardiac lesions.

The second wall L2 is the wall delimiting the myocardium and the left ventricle VG. The first wall L1 surrounding the second wall L2 is the outer wall, namely facing outwardly of the left ventricle VG, of the part of the myocardium surrounding the left ventricle VG. This is the epicardium.

The second wall L2 is the myocardial wall delimiting the left ventricle. This is the endocardium.

In the images of FIG. 6, which are cross-sections of the heart, these walls L1, L2 form closed curves in that they completely surround the left ventricle VG in minor-axis cross-sections.

It is easy to understand that in 3D these walls form surfaces.

Alternatively, segmenting SEG is implemented so as to generate positioning data of only one of these two walls, for example of the outer wall of the myocardium.

Segmenting is performed by implementing a learning function or algorithm, for example an artificial neural network, to segment a bright blood image so as to delimit at least one wall of the myocardium surrounding and delimiting a cavity of the heart.

In a non-limiting example, the learning function is a neural network. The artificial neural network used for segmenting is advantageously a convolutional neural network.

The convolutional neural network is, for example, of the U-Net type or of the transformer type also called self-attention model, for example, of the type commonly called swin transformer.

The neural network, or more generally the learning function, is implemented on two-dimensional images (2D) and/or on three-dimensional images (3D). Stated differently, it is trained to perform the desired segmentation by receiving 2D and/or 3D images as an input.

Advantageously, the learning function is trained, prior to implementing the method according to the invention, from bright blood images of the heart, generated from signals acquired during respective bright blood acquisition steps distinct from inversion-recovery sequences, and labeled by specialists, namely segmented by specialists, so that the trained learning function receiving input data comprising a bright blood image of the heart, is able to segment so as to delimit at least one wall of the myocardium surrounding and delimiting a cavity of the heart.

The learning function is, for example, configured to deliver, from a bright blood input image generated from signals acquired during a bright blood acquisition step distinct from an inversion-recovery sequence, an output image in which the pixels or voxels corresponding to the walls or contours L1 and L2 are colored in a predetermined intensity or color or in respective predetermined colors.

### Propagation

The method may comprise a step of propagating the walls detected during the segmentation step SEG, on at least one black blood image. Stated differently, the method may comprise a step of transferring REP, namely propagating, comprising, identifying, on a black blood image ISNₖ, pixels or voxels corresponding to the walls L1 and L2 identified upon segmenting SEG.

In FIG. 6, a black blood image ISNₖ of the heart cross-section has schematically been represented at the bottom left and at the bottom right the black blood image on which the walls L1 and L2 detected during the segmentation step SEG has been represented in thick black lines.

Identifying, on the black blood image ISNₖ, the pixels or voxels corresponding to the first wall L1 and respectively to the second wall L2 is determined from the positions of the pixels or voxels corresponding to these walls on the bright blood image ISBₖ.

These pixels or voxels may have the same respective positions on the bright blood image and on the black blood image when considering that these images are spatially registered and because these images have the same size and same resolution.

A predetermined or calculated spatial shift may alternatively be applied to these pixels or voxels when it is felt that a spatial shift exists between these images.

Transferring may comprise annotating or colorizing the pixels or voxels corresponding to the walls L1 and L2. Advantageously, characterizing comprises segmenting each bright blood image ISBₖ so as to generate respective positioning data obtained from the respective bright blood image ISBₖ.

Advantageously, characterizing comprises transferring on each black blood image ISNₖ, pixels or voxels corresponding to the walls L1 and L2 identified upon segmenting SEG, from one of the bright blood images ISBₖ. The pixels or voxels transferred on the different black blood images or combined black blood images are advantageously identified from respective bright blood images.

Advantageously, the positioning data used for transferring on a black blood image ISNₖ are generated from a bright blood image ISBₖ generated from signals measured during a same elementary acquisition sequence.

Thus, the positioning data generated from a k-order combined bright blood image ISBₖ are advantageously transferred to a k-order combined black blood image ISNₖ.

### Lesion Characterization Step

The lesion characterization step CAR consists in characterizing the heart from the lesion point of view using one or more black blood images ISNₖ and positioning data of at least one wall, for example the second wall L2, obtained from one or more bright blood images ISBₖ.

This step advantageously makes it possible to generate data characterizing the heart from the lesion point of view.

This step is implemented by the processing unit CT.

The characterization step CAR may comprise a lesion detection step DE and/or a lesion characterization step CAE.

Advantageously, when a lesion is detected, the lesion characterization step CAE is implemented. Stated differently, the lesion characterization step CAE can be implemented only provided that a lesion is detected during the detection step DE.

Alternatively, the detection step DE is implemented after the lesion characterization step CAE or after one of the steps of this actual lesion characterization step CAE.

Alternatively, the lesion characterization step CAE is free of a detection step DE.

The detection step will be described subsequently.

The lesion characterization step CAE advantageously comprises a for of lesionally segmenting SC at least one black blood image ISNₖ so as to generate positioning data of at least one cardiac lesion possibly present in the figure, lesional segmenting SC using positioning data of the first wall L1 and possibly those of the second wall L2, generated during the segmentation step SEG.

The lesion characterization step CAE possibly comprises the following steps:

calculating CTA lesion size CIC from the positioning data of the first wall L1 and possibly of the second wall L2 derived from the segmentation step SEG, and/or
calculating CTT of at least one transmurality degree TR of the lesion CIC from the first wall L1 and the second wall L2 derived from the segmentation step SEG.

These calculations are performed using a set of at least one black blood image.

The size of a lesion is understood to mean a piece of data representative of dimensions of the lesion, such as a volume or a surface area, for example, or a number of pixels or voxels.

### Lesional Segmentation

The lesional segmentation SC uses one or more black blood image(s) ISNₖ and positioning data of the first wall L1 and possibly those of the second wall L2 derived from segmentation SEG.

These positioning data can be positioning data generated during the segmentation step SEG or positioning data derived from the transfer step REP. Alternatively, the lesional segmentation step SG comprises the transfer step.

This step enables cardiac lesions to be located, namely location data of the cardiac lesions to be generated.

Such lesion location data comprises for example the identification or positions of pixels or voxels corresponding to lesions.

The lesional segmentation step SC is advantageously implemented by thresholding.

It advantageously comprises identifying pixels or voxels having an intensity greater than or equal to a predetermined intensity threshold only in a predetermined zone of at least one black blood image ISNₖ delimited by the first wall L1 and/or the second wall L2. Indeed, as can be deduced from FIG. 2, the lesions have, on the black blood images, a high intensity compared to healthy myocardium and blood.

This zone is determined from the positioning data of the first wall L1 and possibly those of the second wall L2 derived from segmentation SEG.

This is for example the zone of a black blood image ISNₖ delimited by the pixels or voxels of the first wall L1 and/or the pixels or voxels of the second wall L2 transferred to the black blood image ISNₖ.

Advantageously, the zone of the black blood image is the zone surrounded and delimited by the first wall L1.

Stated differently, the lesional segmentation step SC comprises searching for pixels or voxels having intensity greater than or equal to a predetermined intensity threshold only in the zone delimited and surrounded by the first wall L1 on one or more black blood image(s) ISNₖ. Stated differently, these pixels or voxels are taken only from the pixels or voxels of a zone of the black blood image(s) surrounded and delimited by the first wall L1. This helps to avoid false detection of lesions beyond the epicardium, avoiding confusion between lesions and fat surrounding the epicardium and represented, on black blood images, by high intensity pixels.

Alternatively, the zone Z is the zone of the black blood image(s) ISNₖ delimited by the first wall L1 and by the second wall L2.

Stated differently, lesional segmentation SC comprises searching for pixels having intensity greater than or equal to a predetermined intensity threshold only in the zone delimited by the two walls L1 and L2 of one or more black blood images ISNₖ. This alternative has the advantage of identifying pixels or voxels of myocardial lesions only. Indeed, it happens that patients have papillary muscle necroses (situated in the zone delimited by the wall L2). In these patients, the muscles are white on the black blood image, which can lead to lesion characterization errors when segmenting the lesions throughout the zone delimited by L1.

Alternatively and/or in addition, the lesional segmentation SC comprises searching for pixels having intensity greater than or equal to a predetermined intensity threshold only in the zone surrounded by the wall L2. This step enables pixels or voxels of papillary muscles only to be identified.

Alternatively, the segmentation is implemented using a neural network, for example, a convolutional neural network trained to segment cardiac lesions in a zone delimited by the walls L1 and/or L2 when receiving as an input the positioning data of the corresponding wall(s) and the black blood image, or using at least one active contour segmentation algorithm, namely a segmentation algorithm using an active contour model.

The detection step DE comprises detecting the absence or presence of lesions using a black blood image ISNₖ and positioning data of at least one wall, for example, of the second wall L2. It outputs an indication of the presence or absence of cardiac lesions.

The detection step DE may be performed by thresholding or using a neural network such as the segmentation step. This step may consist in determining whether a number of contiguous pixels or voxels greater than a predetermined threshold has an intensity greater than a predetermined threshold in the zone delimited by the wall L1 and/or the wall L2 whose positioning is defined during the myocardial segmentation step SEG. The presence of cardiac lesions is detected if this condition is met and the absence of cardiac lesions is detected if this condition is not met.

The neural network is, for example, a convolutional neural network. The neural network is, for example, trained to detect the presence or absence of cardiac lesions in a zone delimited by the walls L1 and/or L2 when it receives as an input the positioning data of the corresponding wall(s) and the black blood image.

Advantageously, when no lesion is detected, namely when the absence of lesion is detected during the step DE.

If no lesion is detected, the next step is advantageously the generation step GENR.

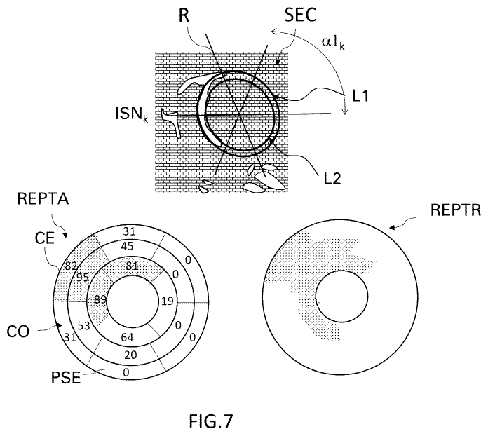

In FIG. 7, the black blood image ISNₖ has been represented, on which the limits L1 and L2 identified upon segmenting and transferred, namely propagated, on the black blood image ISNₖ as well as the pixels identified as being pixels of the lesion have been represented in thick lines.

### Lesional Size Calculation

Characterizing CAR advantageously comprises a step of calculating CTA lesional size.

This calculation step CTA comprises determining at least one elementary piece of data representative of the size of at least one cardiac lesion, for example of the myocardium, by using location data of the location data of the first and/or second walls L1, L2 which are for example directly the positioning data derived from segmenting SEG the myocardium or data derived from these data, for example, data derived from the transfer step or lesion positioning data obtained during the lesional segmentation step SC.

An elementary piece of data representative of a lesion size may be a percentage of a surface area of the myocardium occupied by a lesion on a sector SEC of a black blood image ISNₖ or a volume or mass of the lesion in this sector SEC starting from the axis I parallel to the axis p and passing substantially through the center of the cardiac cavity on the black blood image ISNₖ and delimited by two rays R starting from the axis I as visible on the black blood image ISNₖ.

The percentage of the zone of the myocardium occupied by the lesion on the sector SEC can be calculated from the ratio of the number of pixels corresponding to the lesion in this sector SEC to the number of pixels corresponding to the myocardium in this sector SEC.

The number of pixels corresponding to the lesion in this sector SEC may be calculated from the location data obtained during the lesional segmentation step or may be directly calculated, during the calculation step CTA, for example by selecting, by thresholding, the number of pixels having an intensity greater than a predetermined threshold in the portion of the sector SEC delimited by the walls L1 and L2 or by the wall L1. The step CTA may comprise calculating a piece of data representative of the lesional size in an sector SEC from several elementary data representative of the lesional size calculated, in this sector SEC, for several black blood images ISNₖ distributed along the axis p.

A combination or an average of the elementary data is for example calculated.

The volume of the lesion on a sector SEC can be calculated from the ratio of the number of pixels corresponding to the lesion on this sector to the number of pixels corresponding to the myocardium on this sector, from the thickness of the cross-section corresponding to a black blood image ISNₖ, when the image is two-dimensional.

The size and/or volume are also advantageously calculated from the predetermined resolution of the images.

It should be noted that the myocardium density is 1.06 g/mL. It is therefore considered that the mass of a lesion is substantially equal to the volume thereof, which makes it possible to evaluate the mass of the lesion.

The calculation step CTA may, for example, comprise dividing the black blood image ISNₖ into a first predefined number, equal to 12 in the non-limiting example of FIG. 1, of predefined sectors SEC of the same α1ₖ opening angle pointing toward the axis I and calculating the percentage of the surface areas of the myocardium occupied by the lesion over the different sectors SEC.

The first number of sectors and the opening angle α1ₖ can vary as a function of the sectional plane PCₖ. For example, as the sectional plane PCₖ gets closer to the apex along the major axis, the number of sectors decreases and the opening angle α1ₖ increases.

This step can be implemented for different black blood images ISNₖ of different cross-sections centered on respective sectional planes PCₖ with k=1 to K. The method advantageously comprises a step of generating GENR, by a computer, for example by the processing unit, a set of at least one representation of lesional characterization data and a step of displaying AFFD at least one representation of the set of at least one representation on a screen of the human-machine interface.

The generation step comprises, for example, generating a piece of data representative of the result of the detection step DE, namely the absence or presence of a lesion, and the display step comprises displaying this piece of data.

The set of at least one representation advantageously comprises a first representation REPT of the (piece of) data representative of the lesional size calculated during the calculation step CTA.

As visible in FIG. 7, a Bull's eye representation of the percentages or data representative of the percentages of the surface areas of the myocardium occupied by the lesion in different sectors of black blood images ISNₖ taken along the respective sectional planes PCₖ can for example be generated.

The Bull's eye representation is defined by the American Heart Association AHA and described in the following article: “Standardized Myocardial Segmentation and Nomenclature for Tomographic Imaging of the Heart: A Statement for Healthcare Professionals From the Cardiac Imaging Committee of the Council on Clinical Cardiology of the American Heart Association. Manuel D. Cerqueira et Al, Circulation, 2002; 105:539-42.

The Bull's eye type representation comprises a plurality of concentric circles CE separated two by two by crowns CO. Each crown CO is assigned to a cross-section or set of contiguous cross-sections, considering that the closer the crown CO corresponds to a cross-section or set of cross-sections near the apex, the closer it is to the center of the circles. Each crown CO is divided into sector portions PSE in which there are displayed, as in the example of FIG. 7, the percentages of the myocardial surface area occupied by a lesion and calculated for the respective sectors of the black blood image ISNₖ of the corresponding cross-section or combinations, for example averages, of the percentages calculated from the percentages of the myocardial surface area occupied by a lesion calculated for the sectors of the black blood images of the set of corresponding cross-sections.

Alternatively and/or additionally, the intensity of the pixels of the different crown portions depends on the percentage calculated. The lower this percentage, the higher the intensity of the corresponding crown.

For example, in FIG. 7, the respective averages of the percentages of the lesion size calculated in the respective sectors defined on three sets of contiguous black blood images distributed along the axis p associated with the respective three crowns corresponding respectively to a cross-section of the apex (internal crown), the midventricular (middle crown) and the basal zone (external crown) have been represented in the form of a known Bull's eye type representation.

The portions of sectors associated with a percentage greater than 80% are represented by dotted lines and those associated with a percentage less than or equal to 80% are represented in white.

When a 3D image of the heart is generated, the image is advantageously divided into several layers along the axis p and the same data as from 2D images are calculated from these different layers.

### Transmurality

The characterization step CAR advantageously comprises a step of calculating a piece of data representative of a transmurality percentage of a lesion. Percentage transmurality means the percentage of a thickness of the myocardium occupied by a lesion.

This calculation step CTT comprises determining at least one piece of data representative of the transmurality percentage of at least one myocardial lesion by using location data of a lesion and location data of the first and/or second walls L1, L2 derived from the segmentation step.

This piece of data can be a percentage of the thickness of the myocardium occupied by a lesion on a sector of the black blood image ISNₖ starting from the axis I.

The percentage of the thickness of the myocardium occupied by the lesion on the sector can be calculated from the ratio of a number of pixels corresponding to the thickness of the lesion on this sector to the number of pixels corresponding to the thickness of the myocardium on this sector. The number of pixels corresponding to the thickness of the lesion may be a number obtained from the results of the lesional segmentation step SC or be calculated, for example by thresholding, during the calculation step CTT from the positioning data of L1 and possibly of L2 derived from the segmentation step SEG.

The number of pixels corresponding to the thickness of the lesion on a sector may be an average or a maximum number of pixels, corresponding to the thickness of the lesion, calculated at different radii of the sector.

The number of pixels corresponding to the thickness of the myocardium on a sector may be an average or a maximum number of pixels, corresponding to the thickness of the myocardium, calculated at different radii of the sector. These numbers are calculated from positioning data of the walls L1 and L2.

The calculation step CTT may, for example, comprise dividing the black blood image ISNₖ into a second predefined number of sectors having the same α2ₖ opening angle pointing toward the center of the cardiac cavity and calculating the percentage of the surface areas of the myocardium occupied by the lesion on the different sectors.

The second number of sectors and thus the opening angle α2ₖ can vary as a function of the sectional plane PCₖ. For example, as the sectional plane PCₖ gets closer to the apex, the number of sectors decreases and the opening angle α2ₖ increases.

Advantageously, the second number is greater than the first number.

The step CTT may comprise calculating a piece of data representative of the transmurality in a sector from several elementary pieces of data representative of the transmurality calculated in this sector for several black blood images ISNₖ distributed along the axis p.

A combination or an average of the elementary data is for example calculated.

This step can be implemented for different black blood images ISNₖ of different cross-sections centered on respective sectional planes PCₖ.

The step GENR advantageously comprises generating a representation REPTR of data representative of a transmurality percentage. The display step AFFD advantageously comprises displaying this representation.

For example, it is possible to generate a Bull's eye type representation of combinations, for example averages, of percentages of the transmurality of the lesion in sectors of sets of contiguous black blood image ISNₖ taken in the respective sectional planes PCₖ.

The intensity of the pixels of this image advantageously, but not necessarily, represents the transmurality percentage.

For example, in FIG. 7, the display has been represented in the form of a Bull's eye-type representation REPTR of the averages of transmurality percentages of the lesion calculated in the defined sectors on several sets of contiguous black blood images taken in respective sectional planes distributed along the axis p.

As previously, the Bull's eye representation comprises a plurality of sector portions whose intensity corresponds to the combination of the transmurality percentage calculated for this sector portion.

The lower this percentage, the higher the intensity of the corresponding crown. Alternatively and/or additionally, the percentages are displayed in the sector portions.

### Advantages

The solution provided makes it possible to obtain images with sufficient resolution and contrast to accurately detect and characterize lesions in a reliable and reproducible manner.

Furthermore, by separating two important pieces of information, namely the heart anatomy and lesions, on two distinct images, namely the bright blood images and the black blood images, respectively, it enables an automatic method for characterizing lesions to be implemented. This automation enables a significant time and reproducibility saving compared to solutions of prior art.

The black and bright blood acquisition sequence of the method according to the invention requires a relatively short acquisition time, in particular when acquiring signals to generate 2D images that involve little calculation. This advantageously makes it possible to implement the acquisition sequence in apnea breathing and to limit movements of the heart between the images and therefore corrections to be performed, which makes it possible to limit the computational resources and the implementation of the method in real time. This also helps to limit artifacts that impair image readability. These artifacts accentuate the difficulty of reconstructing sharp and accurate images in order to locate and detect the lesion. In addition, long MRI acquisitions are uncomfortable for the patient. A duration of 10 to 20 min is considered as a very long duration and it is difficult for the patient to stay within the MRI without making a movement.

Furthermore, in the case of the generation of 2D images by the method according to the invention, artifacts of an image extracted from a sectional plane of the 3D image likely to lead to cases in which it is impossible to discriminate between the presence of a potential lesion and the presence of blood located in proximity to the muscle are avoided. Indeed, in some cases, the lesion is so close to blood, it is referred to as subendocardial, that it is difficult to know, in images having artifacts, whether it is a lesion, blood or an image artifact.

The method according to the invention makes it possible to base a clinical decision with little risk of diagnostic error on the presence of a lesion or not.

### Hardware

From a hardware point of view, the processing system TC may be seen as a calculator interacting with computer programs.

The processing unit TC comprises a least a computer, for example, a microcomputer, a network of computers, an electronic component, a tablet, a smartphone, or a Personal Digital Assistant (PDA).

The processing and control unit TC comprises, for example, a calculator, comprising a set of at least one processor, and possibly a memory operably coupled to the calculator.

The memory comprises, for example, a computer-readable medium. The computer-readable medium is a tangible device readable by a reader of the processing unit, able to store electronic instructions and to be coupled to the communication means or communication unit.

Stated differently, the machine-readable medium is a tangible medium. Stated differently, it is not a transient signal per se, such as radio waves or other freely propagating electromagnetic waves, such as light pulses or electronic signals. Such a computer-readable storage medium is, for example, an electronic storage device, a magnetic storage device, an optical storage device, an electromagnetic storage device, a semiconductor storage device, or any combination thereof.

By way of example, the readable medium is an optical disc, a magneto-optical disc, a ROM (Read-Only Memory), an EPROM (Erasable Programmable Read-Only Memory), an EEPROM (Electrically Erasable Programmable Read-Only Memory), a RAM (Random Access Memory), a magnetic card or an optical card.

The readable medium may include an operating system and load the programs according to the invention. It includes registers adapted to record parameter variables created and modified during the execution of the aforementioned programs. A computer program including software instructions is then stored on the readable medium.

Alternatively, the program instructions are derived from an external source and downloaded via a network. This is notably the case for the applications.

The processing and control unit comprises a calculator, namely at least one data processing electronic circuit designed to manipulate and/or transform data represented by electronic or physical quantities in registers of the evaluation system and/or memories into other similar data corresponding to physical data in the register memories or other types of display devices, transmission devices or storage devices.

The processing unit CT comprises, for example, memories, for storing data, for example black blood and bright blood images, operationally coupled to the data processing circuit and a reader adapted to read the computer-readable medium.

The steps of the method according to the invention are, for example, executed by causing the processing circuits of the processing unit CT to read predetermined programs recorded on hardware such as memories such that their data processing circuits execute calculations, control communications and read and/or write data in/to memories.

The characterization is, for example, executed on a processing device, for example a single computer, or on a system distributed between several computers (especially via the use of cloud computing).

The processing unit CT comprises at least one calculator comprising at least the elements listed below: a set of one or more processors (e.g. a central processing unit (CPU), a graphic processing unit (GPU), a microcontroller and/or a digital signal processor (DSP) ASIC able to interpret instructions in the form of a computing program and/or a hardware set such as an application-specific integrated circuit (ASIC), a Field Programmable Gate Array (FPGA), a Programmable Logic Device (PLD), Programmable Logic Arrays (PLA), a System On Chip (SOC), and/or an electronic board in which the steps of the method according to the invention are implemented in hardware elements.

The invention concerns a computer program product comprising the computer-readable medium containing instructions which, when executed by the processing circuit, cause the system S to implement the steps of the method according to the invention.

The program product may comprise the computer-readable recording medium.

The invention also concerns a computer-readable medium, having the computer program recorded thereon.

Alternatively, the program instructions are derived from an external source and downloaded via a network. This is notably the case for the applications. In this case, the computer program product comprises a computer-readable data medium on which program instructions are stored or a data medium signal on which the program instructions are coded.

The form of the program instructions is, for example, a form of source code, a computer-executable form, or any form intermediate between source code and a computer-executable form, such as the form resulting from the conversion of the source code via an interpreter, assembler, compiler, link editor, or locator. Alternatively, the program instructions are a microcode, firmware instructions, status definition data, configuration data for integrated circuits (e.g. VHDL) or an object code. The program instructions are written in any combination of one or more programming languages, for example an object-oriented programming language (C++, JAVA, Python), a procedural programming language (for example language C).

The communication unit comprises at least one communication device allowing communication between elements of the system and possibly between at least one element of the system and a device external to the system. The communication systems may establish a physical link between elements of the system and/or between an element of the system and a device external to the system and/or a remote (wireless) communication link between elements of the system and/or between an element of the system and a device external to the system.

The communication device may comprise any hardware, firmware and/or software suitable for communicating information between elements of the device to which the communication device belongs, for example via a data bus, or to an element external to the device. In order to enable data communication between different devices to which communication devices belong, where applicable, these devices comprise firmware and/or software for establishing, between them, a wired or wireless communication link, for example Wi-Fi, Bluetooth, cellular or Ethernet.

The user interface INT allows a user to enter data or commands so as to be able to interact with the programs according to the invention.

The user interface INT comprises, for example, an output interface INTS and an input interface INTE.

The input interface comprises, for example, a keyboard or a pointing interface, such as a mouse, an optical pen, a touchpad, a remote control, a voice recognition device, a haptic device.

The output interface INTS is designed to render information to a user, sensorily or electrically, such as, for example, visually or acoustically. The output interface comprises, for example, a display. The display step AFFD may be a step of rendering information by a means other than a display.

The output interface INTS may be the input device INTE, for example, in the case of a touch tablet.

1. A method for heart lesion characterization using images of a zone to be imaged comprising the heart of a patient, the heart comprising a myocardium delimiting a cavity of the heart, the images comprising:
a black blood image generated from signals acquired during a black blood-late gadolinium enhancement magnetic resonance acquisition,
a bright blood image generated from signals acquired during a bright blood-late gadolinium enhancement magnetic resonance acquisition, the bright blood acquisition being distinct from an inversion-recovery sequence,
said method comprising:
computer segmenting the bright blood image so as to generate positioning data of a set of at least one wall delimiting the myocardium,
computer heart lesionally characterizing from the black blood image and positioning data, at least one wall of the set of at least one wall delimiting the myocardium, taken from the positioning data of the set of at least one wall delimiting the myocardium.

2. The method according to claim 1, wherein the computer segmenting uses a learning function to segment the bright blood image so as to obtain the positioning data.

3. The method according to claim 2, wherein the learning function is a convolutional neural network.

4. The method according to claim 2, wherein the learning function is trained from a set of training images of the zone to be imaged which are generated from signals acquired during respective bright blood-late gadolinium enhancement magnetic resonance acquisition steps distinct from inversion-recovery sequences.

5. The method according to claim 1, wherein the set of at least one wall comprises a first wall delimiting and surrounding the myocardium.

6. The method according to claim 5, wherein characterizing comprises lesional segmentation to locate a myocardial lesion on the black blood image by using data derived from positioning data of the first wall

7. The method according to claim 6, wherein the lesional segmentation comprises selecting the pixels of the black blood image having an intensity greater than a predetermined threshold, the pixels being taken only from the pixels of the black blood image surrounded by the first wall.

8. The method according to claim 5, wherein the set of at least one wall comprises a second wall delimiting the myocardium and surrounded by the first wall.

9. The method according to claim 8, wherein characterizing comprises calculating data representative of a transmurality percentage of the lesion from data derived from the positioning data of the first wall and the second wall.

10. The method according to claim 8, wherein characterizing comprises calculating a piece of data representative of a lesion size from data derived from the positioning data of the first wall and the second wall (L2).

11. The method according to claim 1, comprising displaying, on a screen, a representation of data calculated during the characterization step.

12. The method according to claim 1, comprising:
black blood-late gadolinium enhancement magnetic resonance acquisition,
generating the black blood image from the signals acquired during the black blood-late gadolinium enhancement magnetic resonance acquisition,
bright blood-late gadolinium enhancement magnetic resonance acquisition,
generating the bright blood image from the signals acquired during the bright blood-late gadolinium enhancement magnetic resonance acquisition.

13. The method of claim 1, wherein the black blood image and the bright blood image are two-dimensional.

14. A system comprising the hardware and software elements to implement the method according to claim 1, the system comprising a processing unit configured to implement the segmentation step and the lesion characterization step.

15. A non-transitory computer program product comprising instructions that cause a system to execute the steps of the method according to claim 1.

16. A non-transitory computer-readable medium, comprising instructions to perform the method according to claim 15.
